(19) United States
(12) Patent Application Publication
(10) Pub. No.: US 2008/0027357 A1
(43) Pub. Date: Jan. 31, 2008

(54) Method for managing labor and childbirth

(76) Inventor: Lydi R. Owen, Las Vegas, NV (US)

Correspondence Address: WEISS & MOY PC, 4204 NORTH BROWN AVENUE, SCOTTSDALE, AZ, 85251, US

(21) Appl. No.: 11/494,139

(22) Filed: Jul. 27, 2006

(51) Int. Cl. A61B 5/103

(52) U.S. Cl. 600/588

(57) ABSTRACT

A method for managing labor and childbirth allows a laboring woman to push prior to full dilation and helps to prevent extreme pain, prolonged labor, and unnecessary cesarean sections and epidural anesthesia. The method involves the initial step of encouraging a laboring woman to wait, without pushing, through first and second stages of labor, culminating in cervical dilation of about five centimeters. It further involves the step of encouraging a laboring woman, during a third stage of labor beginning at a cervical dilation of about five to six centimeters and before a cervical dilation of ten centimeters, of pushing in response to an urge to push. In some embodiments, breathing and positioning techniques are utilized to increase the comfort of the birthing process.

### FIELD OF THE INVENTION

This invention relates generally to methods for labor and childbirth and, more particularly, to a method for managing labor and childbirth in which a laboring woman is encouraged to push when she feels an urge to do so; and that helps to prevent extreme pain, prolonged labor, and unnecessary cesarean sections and epidural anesthesia

### BACKGROUND OF THE INVENTION

There is a rule in obstetrics which currently states that a laboring woman must not be allowed to push until her cervix is completely dilated to 10 centimeters (the “rule of ten”). A laboring woman is routinely told by doctors, nurses, midwives, and doulas that if she pushes before dilation is complete (regardless of the fact that most women have an irresistible urge to do so), she will tear the cervix or cause it to swell and thereby injure herself and/or injure or cause the death of her unborn child.

During the labor process, the cervix undergoes physiological changes. It is generally taught in childbirth education classes, medical schools, and midwifery schools that there are tree stages of labor, including the following: stage one, in which the cervix becomes completely dilated; stage two, in which the infant is delivered; and stage three, in which the placenta is delivered.

In the past, doctors and midwives would encourage a laboring woman to begin pushing early on in labor, before the cervix would become soft and stretchy, and before the laboring woman would have a natural urge to push. Such a practice was likely based on a gross lack of understanding of the physiology of labor and childbirth. This practice increased the risk of injury to the cervix and the unborn child because it is unnatural, and under normal circumstances, most women would have no natural urge to push at such an early stage. When this became evident to doctors and midwives, the rule of ten was established. Under current obstetric standards, pushing is generally not allowed until the cervix is completely dilated. However, this practice causes pushing to occur at a later stage than is optimal.

It is illogical to ask a laboring woman to ignore the need to bear down, the very clear signal from her baby and her body that she should begin to help her baby rotate through the pelvis and in the process to complete the dilation process very quickly. (This can also be life-saving for the unborn child who is tightly wrapped in his/her umbilical cord.) The upward traction exerted by the retracted muscle fibers in the upper uterine segment pulls on the margin of the weakened area, the cervix, and causes it to open. However, without pressure applied from within the uterus by the baby, dilation will not proceed normally. The infant's well-flexed head will, when closely applied to the cervix, aid in dilation. Although the uterus and baby are both pushing at this stage, under current obstetric standards, the mother is instructed not to push when she has not yet dilated to 10 centimeters. In order to avoid pushing when she feels an urge to push, the mother is typically instructed to pant, until the uterus and baby have done all of the work for her. This means harder work for the baby to make its rotations through the curves of the pelvis unaided, and often results in the mother demanding epidural anesthesia because she is not allowed to work with the intensity of the contractions, making her pain unbearable.

A need therefore exists for a method for managing labor and childbirth in which the mother is encouraged to push when she feels an urge to do so, and that helps to prevent extreme pain, prolonged labor, and unnecessary cesarean sections and epidural anesthesia.

The present invention satisfies these needs and provides other, related advantages.

### SUMMARY OF THE INVENTION

In accordance with an embodiment of the present invention, a method for managing labor and childbirth is disclosed. The method comprises the steps of: allowing a laboring woman to proceed naturally through a first stage of labor, defined as occurring when the laboring woman's cervix has become flattened and dilated to approximately one to three centimeters; allowing the laboring woman to proceed naturally through a second stage of labor, defined as occurring from an onset of contractions of active labor until the laboring woman's cervix has achieved dilation of approximately five centimeters, including encouraging the laboring woman to not push during the second stage; encouraging the laboring woman to push, in response to an urge to push, during a third stage of labor, defined as occurring after the second stage of labor, when there is an abrupt change in an intensity of the laboring woman's contractions and when dilation proceeds rapidly, but before dilation has reached ten centimeters; and encouraging the laboring woman to push, during a fourth stage of labor, after dilation has reached ten centimeters.

In accordance with another embodiment of the present invention, a method for managing labor and childbirth is disclosed. The method comprises the steps of; allowing a laboring woman to proceed naturally through a first stage of labor, defined as occurring when the laboring woman's cervix has become flattened and dilated to approximately one to three centimeters; allowing the laboring woman to proceed naturally through a second stage of labor, defined as occurring from an onset of contractions of active labor until the laboring woman's cervix has achieved dilation of approximately five centimeters, including encouraging the laboring woman to not push during the second stage; encouraging the laboring woman to push, in response to an urge to push, during a third stage of labor, defined as occurring after the second stage of labor, when there is an abrupt change in an intensity of the laboring woman's contractions and when dilation proceeds rapidly, but before dilation has reached ten centimeters; encouraging the laboring woman, during the third stage of labor, to engage in a breathing technique having the following steps: the laboring woman takes in a deep breath at the start of a contraction and then blows it out; the laboring woman takes in another deep breath and holds it for at least one second while gently beginning to bear down with her baby for at least one second and then blows the breath out; and the laboring woman continuing the previous two steps until the contraction subsides; and encouraging the laboring woman to push, during a fourth stage of labor, after dilation has reached ten centimeters.

In accordance with a further embodiment of the present invention, a method for managing labor and childbirth is disclosed. The method comprises the steps of: allowing a laboring woman to proceed naturally through a first stage of labor, defined as occurring when the laboring woman's cervix has become flattened and dilated to approximately one to three centimeters, including encouraging the laboring woman to engage in deep breathing and relaxation techniques; allowing the laboring woman to proceed naturally through a second stage of labor, defined as occurring from an onset of contractions of active labor until the laboring woman's cervix has achieved dilation of approximately five centimeters, including encouraging the laboring woman to not push during the second stage and to engage in deep breathing and relaxation techniques; encouraging the laboring woman to push, in response to an urge to push, during a third stage of labor, defined as occurring after the second stage of labor, when there is an abrupt change in an intensity of the laboring woman's contractions and when dilation proceeds rapidly, but before dilation has reached ten centimeters; encouraging the laboring woman, during the third stage of labor, to engage in a breathing technique having the following steps: the laboring woman takes in a deep breath at the start of a contraction and then blows it out; the laboring woman takes in another deep breath and holds it for at least one second while gently beginning to bear down with her baby for at least one second and then blows the breath out; and the laboring woman continuing the previous two steps until the contraction subsides; encouraging the laboring woman to push, during a fourth stage of labor, after dilation has reached ten centimeters; positioning the laboring woman in one of a lying down position and a semi-sitting position on a floor, wherein the laboring woman is propped up with pillows at approximately a 45-degree angle, during the fourth stage of labor; and allowing the laboring woman to proceed naturally through a fifth stage of labor, defined as occurring when a placenta of the laboring woman is delivered, prior to an umbilical cord being cut.

### BRIEF DESCRIPTION OF THE DRAWINGS

FIG. 1 is a flowchart showing the steps involved in an embodiment of the present invention.

FIG. 2 is a flowchart showing the steps involved in an embodiment of the present invention.

### DETAILED DESCRIPTION OF THE PREFERRED EMBODIMENTS

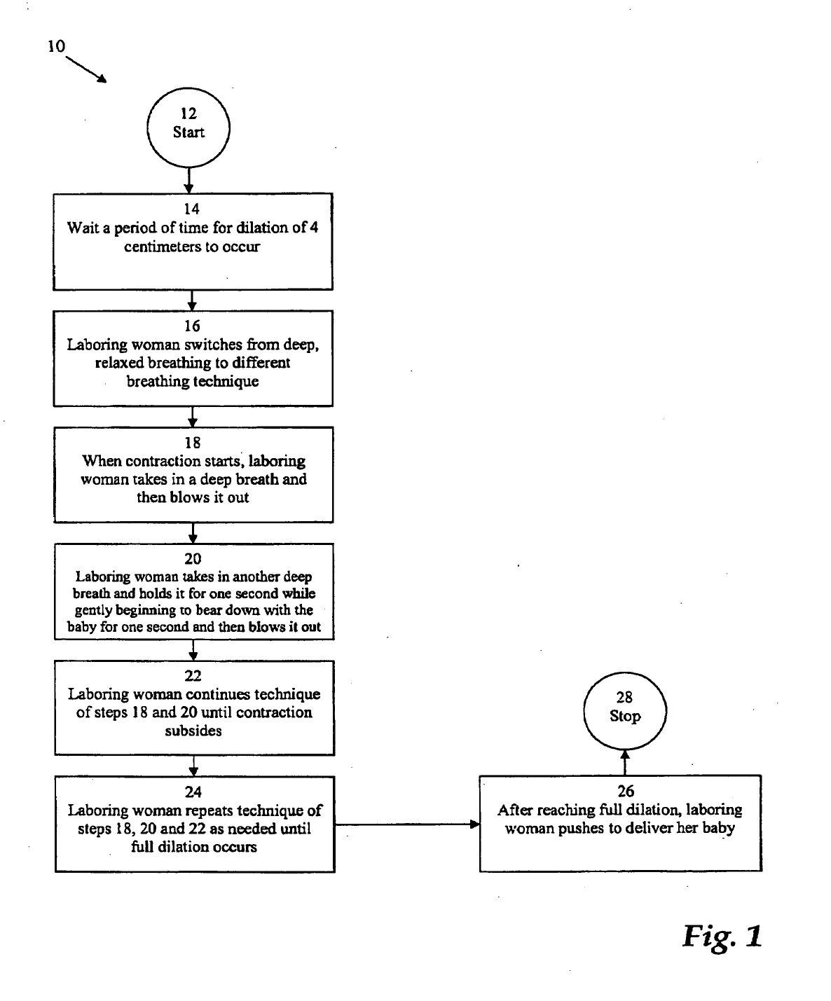

Referring first to FIG. 1, a flowchart shows the steps of a method for managing labor and childbirth 10 (hereinafter the “method 10”), consistent with an embodiment of the present invention. The method 10 is useful for preventing extreme pain, prolonged labor, and unnecessary cesarean sections and epidural anesthesia during childbirth. The present invention dispenses with a general rule in obstetrics which currently holds that a laboring woman must not be allowed to push until her cervix is completely dilated to ten centimeters. Rather with the present invention, the laboring woman is encouraged to push when she feels a natural urge to do so.

A preferred embodiment of the method 10 comprises several steps. The first step of the method 10 involves waiting a period of time for cervical dilation of 4 centimeters to occur 14. Prior to this time, the laboring woman will have gone through a first stage of labor, preferably defined as the softening and effacement of the cervix, which naturally occurs in pregnant women during the last week of pregnancy. During this time, the cervix preferably will have gone from a thick state of approximately 2.5 centimeters to a flattened state in which the cervix is dilated approximately 1-3 centimeters. The lower uterine segment is usually V-shaped prior to labor, when the cervix is not yet dilated, and the baby's head is generally just dipping into the pelvic cavity. When the cervix is totally effaced and the ligaments of the pubic bone have had time to soften and open, the head of the baby is able to descend into the pelvic cavity and the lower uterine segment becomes U-shaped.

At this point, the laboring woman should be ready to proceed to a second stage of labor, preferably defined as that which occurs from the onset of contractions of active labor until 5 centimeters dilation. During this time, the cervix generally is very thin and stretchy and should yield easily to the baby's attempt to rotate through the pelvis. Most laboring women remain dilated at 4 centimeters for the majority of their labor time, until the cervix changes from a leathery-like state into a much softer state. At such time, the cervix generally should be ready to begin opening rapidly by the pressure exerted against it by the baby as it attempts to be born. During this second stage of labor, no pushing is required by the laboring woman. Preferably, she should not be encouraged to push, and generally, she will not have a desire to push because she may feel pain in doing so. The second stage of labor may last anywhere from 1-40 hours, depending on the shape of the laboring woman's pelvis in relation to the baby's bead size and the laboring woman's readiness for labor, childbirth, and becoming a parent.

It should be understood that this second stage of labor may last for many hours without any significant cervical dilation. Many doctors and nurses typically may refer to this lack of cervical dilation as “failure to progress” and perform an unnecessary cesarean section at this stage. In hospitals, when a laboring woman is 3 centimeters dilated, she is generally expected to dilate 1 centimeter every hour thereafter. If her body fails to comply, which it often does, she will either be given a labor-inducing drug, such as Pitocin, or the baby will be delivered by cesarean section. By waiting and allowing the laboring woman's cervix to naturally reach 4 centimeters dilation, unnecessary cesarean sections and labor-inducing drug usage may be avoided.

It should be noted that there is a physiological reason why the cervix stays dilated at 4 centimeters for as long as it does under normal circumstances. Each contraction starts in the upper region of the uterus and spreads downward, being stronger and persisting longer in the upper region. On reaching the lower uterine segment, the contraction weakens considerably, permitting the cervix to dilate. There is neuromuscular harmony between the upper and lower segment of the uterus throughout labor. The muscle fibers of the upper segment contract strongly and retract while the fibers of the lower segment contract only slightly and dilate. The upper segment eventually increases in thickness up to four times its original size, diminishing its cavity significantly. As this is happening, the lower segment—a thin, stretchy area formed by the neck of the cervix that is 7.5 to 10 centimeters long—becomes more and more yielding to the pressure of the baby's head against it. This is a major protection for the unborn baby because as the cavity diminishes in size and the lower segment becomes thin, the baby drops further and further into the pelvis without changing its position in relation to the placenta and umbilical cord. Many babies are wrapped in their umbilical cords because they are active and manage to get tangled up in them at some point during the pregnancy. If the decrease in the size of the cavity did not take place, many babies would be seriously compromised and could easily get strangled by their umbilical cords. Once the laboring woman's cervix reaches dilation of 4 centimeters, preferably, she is allowed to remain dilated at 4 centimeters as long as necessary, in order for her body to adjust and prepare for the next phase of labor. This is normal and natural for most women. Up to this point, the baby's head has been descending and flexing, but no significant degree of rotation has taken place because the cervix has not been thin and yielding enough to allow the baby to begin its descent through the curves of the pelvis.

During the first and second stages of labor, as preferably defined above, and up until the laboring woman's cervix reaches approximately 5-6 centimeters dilation, preferably the laboring woman is engaged in deep breathing and relaxation techniques (as taught by prior art childbirth methods), in order to help her cope with her labor contractions. In the next step of the method 10, the laboring woman switches from deep, relaxed breathing to a different breathing technique 16. This step of switching breathing techniques should occur during a third stage of labor preferably defined by a period in which there is an abrupt change in the intensity of the laboring woman's contractions. At this point, deep, relaxed breathing is no longer effective for most laboring women, because as the cervix lets go of its hold on the unborn baby, dilation proceeds more rapidly, as the baby pushes hard with its feet against the strongly contracting fundus, in order to propel forward while simultaneously being pushed out of the diminished upper uterine segment.

The next step of the method 10 involves a new breathing technique, wherein at the start of a contraction, the laboring woman takes in a deep breath and then blows it out 18. The next step involves the laboring woman taking in another deep breath and holding it for one second, while gently beginning to bear down with her baby for one second and then blowing the breath out 20. In the next step, the laboring woman continues the technique of steps 18 and 20 until the contraction subsides 22. Depending on the strength of a given contraction, it may be desired for the laboring woman to push gently with a first breath, to push more firmly and for a longer duration with a subsequent breath, or to not push at all. The laboring woman should merely be given encouragement to push when she feels a desire to push. In addition, it may also be desired for the laboring woman to sometimes substitute an “oh” sound for a push, or to grunt while pushing. By being allowed to push when she feels a desire to do so, the laboring woman will receive several benefits. For example, by applying pressure to her dilating cervix, it may help to numb her pain, thereby preventing extreme pain typically associated with natural childbirth. In addition, since her mind may only think about one thing at a time (at least consciously), when she is concentrating on working, she may not focus on the pain she may be experiencing. This may help to avoid the need or request for epidural anesthesia. Further, the mother is able to reclaim the power which has been denied her from current obstetric practices. In addition, she is able to imprint with her baby, laying the foundation for a secure and loving ongoing bonding between her and her child.

When the laboring woman is allowed lo bear down when she feels the urge to do so, the cervix is unable to hold the baby back any longer. With the three-fold effort exerted simultaneously by the uterus the baby, and the mother, the cervix quickly progresses from 5-6 centimeters to 10 centimeters dilation (with the amount of time required to do so generally varying from a few minutes to an hour in most instances).

During the third stage of labor, it may be desired for the laboring woman to sit on a toilet, with her legs apart, accompanied by a support person, such as the father of the baby, a midwife, a doctor, a nurse, etc. Preferably, the support person stands in front of the laboring woman during a contraction, holding her hands while the laboring woman bears down.

The next step involves the laboring woman repeating the technique of steps 18, 20, and 22 as needed, until full dilation occurs 24. By employing the technique described in steps 16-24, dilation from 4 to 10 centimeters tends to occur much more rapidly than with standard obstetric practices, thereby helping to avoid prolonged labor. In the final step of the method 10, when the laboring woman's cervix is fully dilated to ten centimeters, she pushes to deliver her baby 26. In reaching full dilation of 10 centimeters, the laboring woman enters into a fourth stage of labor, preferably defined as the actual delivery of the baby.

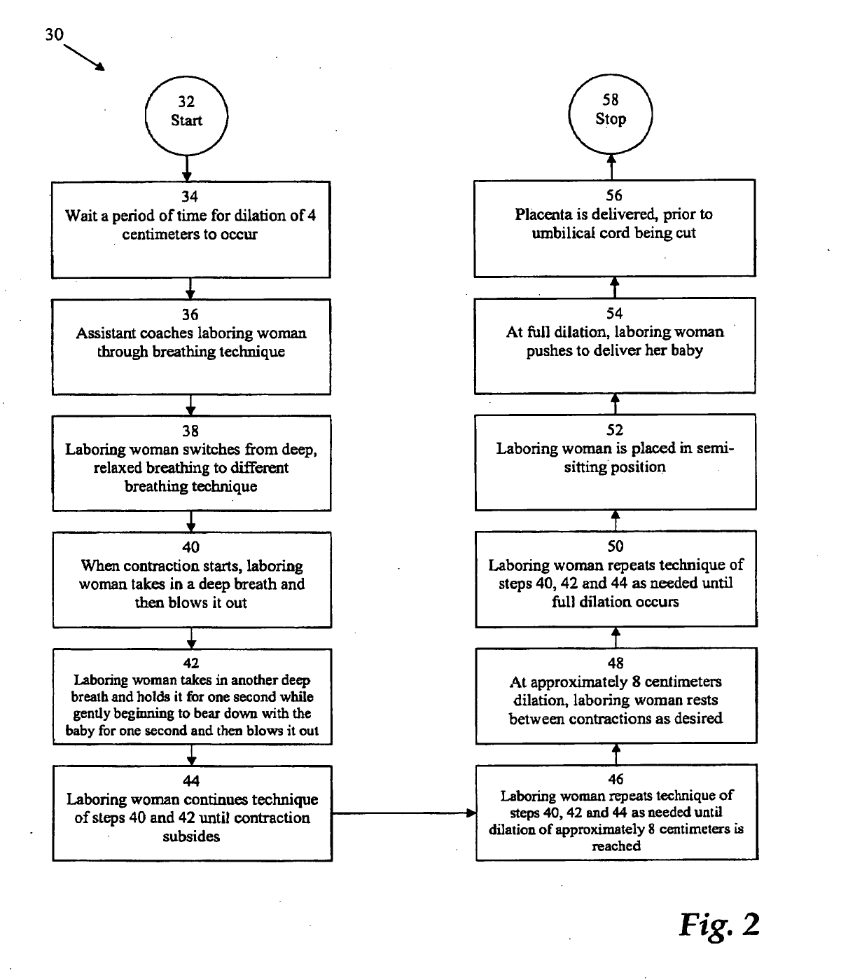

Referring next to FIG. 2, a flowchart shows the steps of a method for managing labor and childbirth 30 (hereafter the “method 30”), consistent with an embodiment of the present invention. As with the method 10, the method 30 is useful for preventing extreme pain, prolonged labor, and unnecessary cesarean sections and epidural anesthesia during childbirth, as described above.

A preferred embodiment of the method 30 comprises several steps. The first step of the method 30 involves waiting a period of time for cervical dilation of 4 centimeters 34 to occur. Prior to this time, the laboring woman will have gone through a first stage of labor and a second stage of labor, as described above with respect to the method 10. During the first and second stages of labor, as preferably defined above, and up until the laboring woman's cervix reaches approximately 5-6 centimeters dilation, preferably the laboring woman is engaged in deep breathing and relaxation techniques (as taught by prior art childbirth methods), in order to help her cope with her labor contractions.

In the next step of the method 30, an assistant coaches the laboring woman through a different breathing technique 36, as next described. In the next step, the laboring woman switches from deep, relaxed breathing to a different breathing technique 38. This step of switching breathing techniques should occur during a third stage of labor, as preferably defined above with respect to the method 10. As discussed above in the method 10, at this point, deep, relaxed breathing is no longer effective for most laboring women. The next step of the method 30 involves a new breathing technique, wherein at the start of a contraction, the laboring woman takes in a deep breath and then blows it out 40. The next step involves the laboring woman taking in another deep breath and holding it for one second, while gently beginning to bear down with her baby for one second and then blowing the breath out 42. In the next step, the laboring woman continues the technique of steps 40 and 42 until the contraction subsides 44. In this embodiment, preferably the laboring woman is coached through several contractions by an assistant such as a doctor, nurse, doula, midwife, husband, partner, etc., at least until she becomes accustomed to the technique of steps 40 and 42 and handling her contractions. Once she becomes accustomed to the technique of steps 40 and 42, the laboring woman may be able to proceed without coaching, as she begins to respond to the contractions instinctively. As with the method 10, depending on the strength of a given contraction, it may be desired for the laboring woman to push gently with a first breath, to push more firmly and for a longer duration with a subsequent breath, or to not push at all. The laboring woman should merely be given encouragement to push when she feels a desire to push. In addition, it may also be desired for the laboring woman to sometimes substitute an “oh” sound for a push, or to grunt while pushing. By being allowed to push when she feels a desire to do so, the Laboring woman will receive several benefits, as discussed above with respect to the method 10.

As with the method 10, during the third stage of labor it may be desired for the laboring woman to sit on a toilet, with her legs apart, accompanied by a support person, such as the father of the baby, a midwife, a doctor, a nurse, etc. Preferably, the support person stands in front of the laboring woman during a contraction, holding her hands while the laboring woman bears down.

The next step of the method 30 involves the laboring woman repeating the technique of steps 40, 42, and 44 as needed, until cervical dilation reaches 8 centimeters 46. When many laboring women reach dilation of 8 centimeters, their endorphins are activated and they become sleepy. At this stage, the laboring woman may wish to lie down. Thus, the next step involves the laboring woman resting between contractions, as desired 48. It may be desired for the laboring woman to take brief naps during this phase. For many laboring women, the natural urge to bear down may diminish for a few minutes, which may be a natural way of allowing the cervix a few moments of reprieve. The next step involves the laboring woman repeating the technique of steps 40, 42, and 44 as needed, until full dilation occurs 50. As discussed with respect to the method 10, by employing the technique described in steps 38-50, dilation from 4 to 10 centimeters tends to occur much more rapidly than with standard obstetric practices, thereby helping to avoid prolonged labor. In the next step of the method 30, the laboring woman is placed in a semi-sitting position on a floor 52. In a preferred embodiment, the laboring woman is positioned on a hard surface, leaning against pillows that have been propped up at approximately a 45-degree angle, The laboring woman preferably pulls her knees up to her chest and outward from her body. While in this embodiment the laboring woman is placed in the semi-sitting position while at full dilation, it may be desired for her to be placed in this position earlier. It may also be desired for the laboring woman to lie down while pulling her knees up to her chest and outward from her body.

In the next step of the method 30, when the laboring woman's cervix is fully dilated to ten centimeters, she pushes to deliver her baby 54. In reaching full dilation of 10 centimeters, the laboring woman enters into a fourth stage of labor, preferably defined as the delivery of the baby. The final step of the method 30 involves the delivery of the placenta, followed by the cutting of the umbilical cord 56. At this point, the laboring woman reaches a fifth stage of labor, preferably defined as the delivery of the placenta. In a preferred embodiment, the placenta is delivered prior to the cutting of the umbilical cord, which helps to minimize bleeding.

While the invention has been particularly shown and described with reference to the preferred embodiments thereof, it will be understood by those skilled in the art that the foregoing and other changes in form and details may be made therein without departing from the spirit and scope of the invention.

I claim:

1. A method for managing labor and childbirth comprising the steps of:
allowing a laboring woman to proceed naturally through a first stage of labor, defined as occurring when the laboring woman's cervix has become flattened and dilated to approximately one to three centimeters;
allowing the laboring woman to proceed naturally through a second stage of labor, defined as occurring from an onset of contractions of active labor until the laboring woman's cervix has achieved dilation of approximately five centimeters, including encouraging the laboring woman to not push during the second stage;
encouraging the laboring woman to push, in response to an urge to push, during a third stage of labor, defined as occurring after the second stage of labor, when there is an abrupt change in an intensity of the laboring woman's contractions and when dilation proceeds rapidly, but before dilation has reached ten centimeters; and
encouraging the laboring woman to push, during a fourth stage of labor, after dilation has reached ten centimeters.

2. The method of claim 1, further comprising encouraging the laboring woman, during the first stage of labor, to engage in deep breathing and relaxation techniques.

3. The method of claim 2, further comprising encouraging the laboring woman, during the second stage of labor, to engage in deep breathing and relaxation techniques.

4. The method of claim 1, further comprising encouraging the laboring woman, during the third stage of labor, to engage in a breathing technique having the following steps:
the laboring woman takes in a deep breath at the start of a contraction and then blows it out;
the laboring woman takes in another deep breath and holds it for at least one second while gently beginning to bear down with her baby for at least one second and then blows the breath out; and
the laboring woman continuing the previous two steps until the contraction subsides.

5. The method of claim 1 wherein the laboring woman is positioned in a semi-sitting position on a floor during the fourth stage of labor.

6. The method of claim 5 wherein the laboring woman is further propped up with pillows at approximately a 45-degree angle.

7. The method of claim 1 wherein the laboring woman is lying down during the fourth stage of labor.

8. The method of claim 5 wherein the laboring woman pulls her knees to her chest.

9. The method of claim 7 wherein the laboring woman pulls her knees to her chest.

10. The method of claim I further comprising a fifth stage of labor, defined as occurring when a placenta of the laboring woman is delivered, prior to an umbilical cord being cut.

11. A method for managing labor and childbirth comprising the steps of:
allowing a laboring woman to proceed naturally through a first stage of labor, defined as occurring when the laboring woman's cervix has become flattened and dilated to approximately one to three centimeters;
allowing the laboring woman to proceed naturally through a second stage of labor, defined as occurring from an onset of contractions of active labor until the laboring woman's cervix has achieved dilation of approximately five centimeters, including encouraging the laboring woman to not push during the second stage;
encouraging the laboring woman to push, in response to an urge to push, during a third stage of labor, defined as occurring after the second stage of labor, when there is an abrupt change in an intensity of the laboring woman's contractions and when dilation proceeds rapidly, but before dilation has reached ten centimeters;
encouraging the laboring woman, during the third stage of labor, to engage in a breathing technique having the following steps:
the laboring woman takes in a deep breath at the start of a contraction and then blows it out;
the laboring woman takes in another deep breath and holds it for at least one second while gently beginning to bear down with her baby for at least one second and then blows the breath out; and
the laboring woman continuing the previous two steps until the contraction subsides; and
encouraging the laboring woman to push, during a fourth stage of labor, after dilation has reached ten centimeters.

12. The method of claim 11, further comprising encouraging the laboring woman, during the first stage of labor, to engage in deep breathing and relaxation techniques.

13. The method of claim 12, further comprising encouraging the laboring woman, during the second stage of labor, to engage in deep breathing and relaxation techniques.

14. The method of claim 11 wherein the laboring woman is positioned in a semi-sitting position on a floor during the fourth stage of labor.

15. The method of claim 14 wherein the laboring woman is further propped up with pillows at approximately a 45-degree angle.

16. The method of claim 11 wherein the laboring woman is lying down during the fourth stage of labor.

17. The method of claim 14 wherein the laboring woman pulls her knees to her chest.

18. The method of claim 16 wherein the laboring woman pulls her knees to her chest.

19. The method of claim 11 further comprising a fifth stage of labor, defined as occurring when a placenta of the laboring woman is delivered, prior to an umbilical cord being cut.

20. A method for managing labor and childbirth comprising the steps of:
allowing a laboring woman to proceed naturally through a first stage of labor, defined as occurring when the laboring woman's cervix has become flattened and dilated to approximately one to three centimeters, including encouraging the laboring woman to engage in deep breathing and relaxation techniques;
allowing the laboring woman to proceed naturally through a second stage of labor, defined as occurring from an onset of contractions of active labor until the laboring woman's cervix has achieved dilation of approximately five centimeters, including encouraging the laboring woman to not push during the second stage and to engage in deep breathing and relaxation techniques;
encouraging the laboring woman to push, in response to an urge to push, during a third stage of labor, defined as occurring after the second stage of labor, when there is an abrupt change in an intensity of the laboring woman's contractions and when dilation proceeds rapidly, but before dilation has reached ten centimeters;
encouraging the laboring woman, during the third stage of labor, to engage in a breathing technique having the following steps:
the laboring woman takes in a deep breath at the start of a contraction and then blows it out;
the laboring woman takes in another deep breath and holds it for at least one second while gently beginning to bear down with her baby for at least one second and then blows the breath out; and
the laboring woman continuing the previous two steps until the contraction subsides;
encouraging the laboring woman to push, during a fourth stage of labor, after dilation has reached ten centimeters;
positioning the laboring woman in one of a lying down position and a semi-sitting position on a floor, wherein the laboring woman is propped up with pillows at approximately a 45-degree angle, during the fourth stage of labor; and
allowing the laboring woman to proceed naturally through a fifth stage of labor, defined as occurring when a placenta of the laboring woman is delivered, prior to an umbilical cord being cut.
